(19) United States
(12) Patent Application Publication
(10) Pub. No.: US 2013/0211503 A1
(43) Pub. Date: Aug. 15, 2013

(54) METHOD OF USING AMNION ALLOGRAFT IN CONGENITAL HEART DISEASE SURGERY

(71) Applicant: AFCELL MEDICAL, (US)

(72) Inventor: Robin R. YOUNG, Wayne, PA (US)

(73) Assignee: AFCELL MEDICAL, Parsippany, NJ (US)

(21) Appl. No.: 13/767,224

(22) Filed: Feb. 14, 2013

Related U.S. Application Data
(60) Provisional application No. 61/598,413, filed on Feb. 14, 2012.

(51) Int. Cl. A61F 2/06

(52) U.S. Cl. CPC A61F 2/06
USPC 623/1.23

(57) ABSTRACT

Improved methods, compositions and kits for congenital heart disease surgeries are described. The methods utilize amniotic fluid and/or an allograft comprising a layer of amnion to improve the performance and reduce complications of the surgeries and the allograft has a pre-made size and shape suitable for the application.

### CROSS-REFERENCE TO RELATED APPLICATIONS

This application is entitled to priority pursuant to 35 U.S.C. §119(e) to U.S. Provisional Patent Application No. 61/598,413, filed Feb. 14, 2012, which is hereby incorporated by reference herein in its entirety.

### BACKGROUND OF THE INVENTION

Congenital heart disease refers to a defect with the structure of the heart or blood vessels due to abnormal heart development before birth. About one in every 120 babies is born with some form of heart defect, and about one million people in the United States have congenital heart disease. There are various kinds of congenital heart diseases or defects, most of which either obstruct blood flow in the heart or vessels near it, or cause blood to flow through the heart in an abnormal pattern. In some rare cases, only one ventricle (single ventricle) is present, both the pulmonary artery and aorta arise from the same ventricle (double outlet ventricle), or the right or left side of the heart is incompletely formed—hypoplastic heart.

Surgeries can be performed to repair congenital heart diseases or defects, including, but not limited to, patent ductus arteriosus (PDA) ligation to surgically close the ductus arteriosus, a shunt connecting the pulmonary artery to the aortic arch in fetus that fails to close it shortly after birth; repair of coarctation of the aortar, a very narrow section on the aorta; repair of atrial septal defect (ASD), which occurs when a natural opening on atrial septum, the wall between the left and right atria (upper chambers) of the heart, fails to close on its own when a baby is born; repair of ventricular septal defect (VSD), a hole in ventricular septum, the wall between the left and right ventricles (lower chambers) of the heart; repair of tetralogy of fallot, a congenital heart defect that usually includes 4 defects in the heart; repair of transposition of the great vessels, when the aorta comes from the left side of the heart, and the pulmonary artery comes from the right side; repair of truncus arteriosus, a rare condition that occurs when the aorta, coronary arteries, and the pulmonary artery all come out of one common trunk; repair of tricuspid atresia, which occurs when tricuspid valve, the valve between the upper and lower chambers on the right side of the heart, is missing; correction of total anomalous pulmonary venous return (TAPVR), when the pulmonary veins bring oxygen-rich blood from the lungs back to the right side of the heart, instead of to the left side of the heart, where it should be; repair of hypoplastic left heart, a very severe heart defect that results from a severely underdeveloped left heart, etc.

Overall mortality related to heart surgeries to repair congenital heart diseases or defects is about 4.0%. Mortality usually occurred in those patients complicated with congestive cardiac failure, lower respiratory tract infection and infective endocarditis. There is a need to improve the performance and reduce risks of heart surgeries to repair congenital heart diseases or defects.

The amnion is a thin, cellular, extra-embryonic membrane that forms the inner membrane of a closed sac surrounding and protecting an embryo in reptiles, birds, and mammals. The sac contains the fetus and amniotic fluid or liquor amnii, in which the embryo is immersed, nourished and protected. Typically, the amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid. The amnion has multiple functions, i.e., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side. The fetal membrane including amnion and chorion has been used in surgeries documented as early as 1910. See Trelford and Trelford-Sauder, The Amnion in Surgery, Past and Present, 134 AM J. OBSTET. GYNECOL 833 (1979). Amnioplastin, an isolated and chemically processed amniotic membrane, was used for continual dural repair, peripheral nerve injuries, conjunctival graft and flexor and tendon repair. See e.g., Chao et al., “A New Method of Preventing Adhesions: the Use of Amnioplastin after Craniotomy,” The British Medical Journal, March 30, 1940. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In 1962, the fetal membrane was used to treat pelvic basins after total exenteration in dogs, however, trials in human proved disappointing.

In recent years, there have been renewed interests in the application of amnion in ocular surface reconstruction, for example, as an allograph for repairing corneal defects. See, for example, Tsai and Tseng, Cornea. 1994 Sep;13(5):389-400; and Dua et al., Br. J. Ophthalmol 1999, 83:748-20 752. In addition, amnion and amniotic fluid have recently been used as sources of placental stem cells. See, e.g., U.S. Pat. No. 7,255,879 and WO 200073421.

The role of the amniotic membrane was investigated in chickens with regard to the prevention of adhesion formation following tendon repair in zone II. Results of histologic examination demonstrated that use of the amniotic membrane significantly reduced the amount of adhesion compared with the other groups. Three months after implantation no remnants of amniotic membrane could be identified at the tendon repair site. Demirkan et al., Archives of Orthopaedic and Trauma Surgery, 2002, vol. 122: 396-399.

Despite the clinical and published record regarding the safety and efficacy of amnion in broad surgical use, issues regarding reproducibility, safety and the precise form of amnion for each prospective indication have prevented amnion from achieving broad commercial distribution.

It has now been discovered that using an allograft comprising an amnion in congenital heart disease surgeries as described in the present invention significantly reduces inflammation and tissue adhesion, promotes uniform re-growth and epithelialization, prevents scar tissue formation, thus significantly improves performance and reduces complications of the congenital heart disease surgeries.

### BRIEF SUMMARY OF THE INVENTION

In one general aspect, the present invention relates to a method of improving a congenital heart disease surgery. In the method, the improvement comprises applying at least one of an allograft and an amniotic fluid over a suture line or an incision or an otherwise injured tissue site resulting from the congenital heart disease surgery, or over or under the pericardium membrane of the subject, wherein the allograft has a pre-made size and shape suitable for the application.

In another general aspect, the present invention relates to an allograft comprising a layer of amnion of a pre-made size and shape suitable for covering a suture line, an incision, or an otherwise damaged tissue site resulting from a congenital heart disease surgery.

In another general aspect, the improvement comprises applying at least one of an amniotic fluid and an allograft comprising a layer of amnion over a skin incision resulting from the congenital heart disease surgery, wherein the allograft has a pre-made size and shape suitable for the application.

Another general aspect of the present invention relates to a kit comprising a plurality of allografts and instructions on how to use the allografts in a congenital heart disease surgery, wherein each of the plurality of allografts comprises a layer of amnion of a pre-made size and shape suitable for covering a suture line or an incision or an otherwise damaged tissue site resulting from the congenital heart disease surgery, or for applying over or under the pericardium membrane of the subject.

Other aspects, features and advantages of the invention will be apparent from the following disclosure, including the detailed description of the invention and its preferred embodiments and the appended claims.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

According to embodiments of the present invention, at least one of an amniotic fluid and an allograft comprising a layer of amnion is used to improve congenital heart disease surgeries. In one embodiment of the present invention, the amniotic fluid and/or allograft is used to improve the performance of an open heart surgery to repair total anomalous pulmonary venous return (TAPVR) as described in detail in the following. Those skilled in the art would readily appreciate that, in view of the present disclosure, similar improvement can also be made to the surgical procedures to repair other congenital heart diseases or disorders, such as PDA, coarctation of the aortar, ASD, VSD, tetralogy of fallot, transposition of the great vessels, truncus arteriosus, tricuspid atresia, hypoplastic left heart, etc.

TAPVR is a congenital heart disease (present at birth) in which none of the four veins that take blood from the lungs to the heart is attached to the left atrium (left upper chamber of the heart). In infants with TAPVR, blood simply circles to and from the lungs and never gets out to the body, i.e., oxygenated blood returns from the lungs back to the right atrium or a vein flowing into the right atrium and not to the left side of heart. Other congenital heart diseases may exist together with TAPVR. For example, a large atrial septal defect (ASD) or patent foramen ovale (passage between the left and right atria) may exist to allow oxygenated blood to flow to the left side of the heart and rest of the body.

Early complete surgical repair is needed for infants with TAPVR to survive. The timing of the surgical repair varies depending on the type of TAPVR present and the condition of the child. Some of these children may actually require extracorporeal life support (ECMO) prior to surgery because of their marked hemodynamic instability. Once symptoms are noted, the child may be admitted to the intensive care unit (ICU) or special care nursery, placed on oxygen, and possibly even on a ventilator, to assist his/her breathing. Intravenous medications may be given to help the heart and lungs function more efficiently.

In virtually all types of TAPVR, the pulmonary veins return to a common confluence behind the left atrium. In a surgery, after opening the heart of the child, the surgeon reconnects the four pulmonary veins to the left atrium. He surgically closes any associated heart defects such as atrial septal defect, ventricular septal defect, patent foramen ovale, and/or patent ductus arteriosus. He also ties off all other routes for pulmonary venous drainage (such as the abnormal vessels which had carried pulmonary vein blood to the supracardiac or infracardiac areas). The operation is performed under general anesthesia.

According to an embodiment of the present invention, at least one of an amniotic fluid and an allograft comprising a layer of amnion is placed over one or more suture lines, incisions or otherwise injured tissue sites resulting from the TAPVR surgery to form a cover and barrier over the suture lines, the incisions or the tissue sites. One or more of allografts comprising a layer of amnion can also be placed over or under the pericardium membrane, e.g., along the anatomical planes, after all defects of TAPVR have been repaired and the pericardial cavity is washed with a saline solution containing one or more anti-microbial agents, such as gentamycin. There are minimal space constraints for the placement of the allograft. Exposure is broad for the entire space.

In one embodiment, a single allograft is used to cover the incisions, suture lines, and otherwise damaged tissue sites resulting from the TAPVR.

In another embodiment, a plurality of allografts, each with the shape and size suitable to cover one or more particular incisions, suture lines or tissue sites, are used to cover different incisions, suture lines, or otherwise damaged tissue sites resulting from the TAPVR.

In an embodiment of the present invention, the allograft to be used to cover the incisions, suture lines or otherwise damaged tissue sites resulting from the congenital heart disease surgery is able to be attached or affixed with fibrin glue, be able to adhere to BioGlue®, or hold a 3.0 or 4.0 prolene, polypropylene or monocryl suture.

The appropriate shape and dimension of the allografts are chosen based on the shape and size of the suture, incision or damaged tissue sites. For example, the allograft to be placed alone anatomical planes under or over the pericardium can have an oval shape, about 3 cm-9 cm in length and about 2 cm-6 cm in width. The allograft to be placed over suture lines can be 1 cm by 2-4 cm.

Preferably, the allograft placed over or under the pericardium and over the sutures and incisions is thin. In one embodiment of the invention, the allograft has a thickness of about 0.02 mm to 0.10 mm. It can have of a single layer of amnion, two layers of amnion, a layer of amnion and a layer of chorion, or a layer of amnion and a layer of other collagen membranes of biological origin. The multiple layers in the allograft can be subjected to a cross-linking treatment to make the layers closely adhere to each other in an integrated form.

In one embodiment of the present invention, the allograft can carry one or more therapeutic agents, such as anti-microbial agents, growth enhancing agent, anti-inflammatory agent, agents that prevent scarring, adhesions and tethering of internal organs and the heart, etc., to further improve the performance and reduce the complications of congenital heart disease surgery. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

The two surfaces of human amnion are structurally different. The surface facing the fetus is smooth and hardly cell adhesive, comprising a thin layer of fine fibers. The surface facing the chorion is rough and suitable for cell proliferation, comprising thick fasciculus. In one embodiment of the present invention, the allograft is placed adjacent to the pericardium so that the chorion facing surface of the amnion faces the suture lines. In another embodiment of the present invention, the allograft is placed adjacent to the pericardium so that the fetus facing surface of the amnion faces the suture lines. The surgeon is provided with a range of sizes and shapes of allograft, such as the diamond shape, the curved cup shape, etc., which can be chosen and oriented according to the size and shape of the patient's anatomy.

In another embodiment of the present invention, an allograft comprising a layer of amnion is used to cover skin incision resulting from the congenital heart disease surgery, e.g., skin incision at sternum, to improve the healing and reduce scar formation. The allograft can be of any size suitable for covering the sutures or other type of tissue injuries at skin incision.

Preferably, a relatively thick layer of allograft is used to cover the skin incision. In one embodiment of the invention, the allograft has a thickness of about 2 mm to 4 mm. It can have multiple layers of amnion or a combination of multiple layers of amnion and chorion.

The present invention overcomes shortcomings of the prior art by using human amniotic fluid and allograft membranes to improve congenital heart disease surgeries.

Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix metalloproteases (MMPs) expressed by infiltrating polymorphonuclear cells and macrophages. Hao et al., 2000, Cornea,19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used to improve the performance of congenital heart disease surgeries.

Amnion used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The present invention encompasses a kit containing allograft for improved congenital heart disease surgeries. The kit comprises allografts, each of which having one or more layers of amnion, and instructions on how to use the allografts in congenital heart disease surgeries. The allograft can also comprise one or more layers of chorion or one or more layers of other collagen membranes of biological origin. The allograft can further comprise one or more therapeutically active agents, such as anti-microbial agents, growth enhancing agents, anti-inflammatory agents and agents which prevent scarring, adhesions and tethering of internal organs and the heart.

Preferably, at least two of the allografts have different sizes and/or thickness.

In one embodiment of the present invention, the kit includes an allograft having a thickness of about 0.02 mm to 0.10 mm, and an oval shape of about 3 cm to 9 cm in length and about 2 cm to 6 cm in width.

In another embodiment of the present invention, the kit further comprises a second allograft comprising a plurality layers of amnion, and optionally one or more layers of chorion, wherein the second allograft has a thickness of about 2 mm to 4 mm, and a rectangular shape of about 10 cm by 5 cm.

In yet another embodiment of the present invention, the kit further comprises an amniotic fluid.

Preferably, all the birth tissues in the kit, e.g., the amnion, chorion and amniotic fluid, are from the same biological source, i.e., the same pregnant woman.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A method of improving a congenital heart disease surgery in a subject, comprising applying at least one of an allograft and an amniotic fluid over a suture line, an incision or an otherwise damaged tissue site resulting from the congenital heart disease surgery, or over or under the pericardium membrane of the subject, wherein the allograft comprises a layer of amnion and has a pre-made size and shape suitable for the application.

2. The method of claim 1, wherein the allograft is attached to a tissue of the subject with a 4.0 suture.

3. The method of claim 1, wherein the allograft has a thickness of about 0.02 mm to 0.10 mm.

4. The method of claim 1, wherein the surgery is an open heart surgery.

5. The method of claim 1, wherein the allograft applied over or under the pericardium membrane has an oval shape, about 3 cm to 9 cm in length and about 2 cm to 6 cm in width.

6. The method of claim 1, wherein the allograft consists of a single layer of amnion, two layers of amnion, or a layer of amnion and a layer of chorion.

7. The method of claim 1, wherein the allograft or amniotic fluid further comprises one or more therapeutically active agents selected from the group consisting of anti-microbial agents, growth enhancing agents, anti-inflammatory agents, and other agents that prevent scarring, adhesions and tethering of internal organs and the heart.

8. The method of claim 1, wherein the amnion or amniotic fluid is obtained using a process comprising:
a. obtaining informed consent from pregnant females;
b. conducting risk assessment on the consented pregnant females to select an amnion donor;
c. procuring after birth placenta from the amnion donor; and
d. obtaining the amnion or amniotic fluid from the placenta.

9. The method of claim 1, further comprising applying at least one of the amniotic fluid and a second allograft comprising a layer of amnion over a skin incision resulting from the congenital heart disease surgery.

10. A method of improving a congenital heart disease surgery in a subject, comprising applying at least one of an amniotic fluid and an allograft comprising a layer of amnion over a skin incision resulting from the congenital heart disease surgery, wherein the allograft has a pre-made size and shape suitable for the application.

11. The method of claim 10, wherein the allograft has a thickness of about 2 mm to 4 mm.

12. The method of claim 10, wherein the allograft comprises multiple layers of amnion and optionally multiple layers of chorion.

13. An allograft comprising a layer of amnion and having a pre-made size and shape suitable for covering a suture line, an incision, or an otherwise damaged tissue site resulting from a congenital heart disease surgery.

14. A kit comprising a plurality of allografts and instructions on how to use the allografts in a congenital heart disease surgery, wherein each of the plurality of allografts comprises a layer of amnion of a pre-made size and shape suitable for covering a suture line, an incision, or an otherwise damaged tissue site resulting from the congenital heart disease surgery, or for covering over or under the pericardium membrane of the subject.

15. The kit of claim 14, wherein the amnion is obtained using a process comprising:
a. obtaining informed consent from pregnant females;
b. conducting risk assessment on the consented pregnant females to select an amnion donor;
c. procuring after birth placenta from the amnion donor; and
d. obtaining the amnion from the placenta.

16. The kit of claim 14, further comprising an amniotic fluid.

17. The kit of claim 14, comprising an allograft having a thickness of about 0.02 mm to 0.10 mm, and an oval shape of about 3 cm to 9 cm in length and about 2 cm to 6 cm in width.

18. The kit of claim 17, further comprising a second allograft comprising a plurality layers of amnion, and optionally one or more layers of chorion, wherein the second allograft has a thickness of about 2 mm to 4 mm, and a rectangular shape of about 10 cm by 5 cm.

19. The kit of claim 18, further comprising an amniotic fluid.
